(19) United States
(12) Patent Application Publication
(10) Pub. No.: US 2025/0064375 A1
(43) Pub. Date: Feb. 27, 2025

(54) DEVICE AND METHOD FOR PERFORMING RECOVERY FUNCTIONS

(71) Applicant: AItewan BioMedical Technology Inc., Taipei City (TW)

(72) Inventors: Yu-Te WU, Taipei City (TW); Fu-Ming WANG, Taipei City (TW)

(21) Appl. No.: 18/807,213

(22) Filed: Aug. 16, 2024

(30) Foreign Application Priority Data
Aug. 25, 2023 (TW) 112132168

(51) Int. Cl. A61B 5/372; A61B 5/00; A61B 5/384

(52) U.S. Cl. CPC A61B 5/372; A61B 5/384; A61B 5/7267; A61B 5/7405

(57) ABSTRACT

A device, used in conjunction with a brainwave detector, includes a display unit configured to display at least one object initial information of an application program, a brainwave signal receiving module for receiving at least one brainwave signal from the brainwave detector, an analysis and judgment unit analyzing and judging the at least one brainwave signal to generate mental activity classification information, and a recover control unit generating alteration information based on the mental activity classification information, recovering the object initial information into object expectation information according to the alteration information, and displaying the object expectation information on the display unit.

### CROSS REFERENCE TO RELATED APPLICATIONS

This Non-provisional application claims priority under 35 U.S.C. § 119(a) on Patent Application No(s). 112132168 filed in Taiwan, Republic of China on Aug. 25, 2023, the entire contents of which are hereby incorporated by reference.

### BACKGROUND

### Technology Field

The present disclosure relates to a method and device for recovering object initial information into object expectation information based on the mental activity of a user. In particular, the present disclosure relates to a method and device for recovering object initial information into object expectation information by analyzing the brainwaves generated based on the mental activity of a user to determine the classification or classification intensity of the mental activity.

### Description of Related Art

In recent years, electroencephalography (EEG) or brain-computer interface (BCI) is a technology that has been widely studied. It has a wide range of applications and is a very potential technology.

EEG technology mainly detects the brainwaves of a subject or user through a brainwave detector, and analyzes the subject's mental activity state through a spectrum analysis model or a signal analysis model. Regarding the spectrum analysis model, it can use the spectrum analysis method to analyze the proportions of different frequency components of the subject's brainwaves to determine the subject's current mental activity state. The mental activity state here refers to the classifications expressed by user's brainwave signals, which include, for example, attention, rest, anxiety and the likes. In general, this analysis can be made by observing the theta waves (4˜8 Hz), alpha waves (8˜13 Hz), and beta waves (13˜30 Hz) produced by the user's frontal lobe. Among the frequency components, theta waves, alpha waves, and beta waves are relevant to rest and attention. Specifically, when the subject is in an attention state, his or her brainwave signals are denser and have more high-frequency components in the spectral components.

In addition to analyzing the subject's mental activity state through spectrum analysis model or signal analysis model, in recent years, some researchers have used machine learning models or deep learning models to detect brainwaves so as to realize the subject's mental activity state. The machine learning model collects, in advance, a large amount of brainwave data of attention and rest, and uses these collected data to train a classifier to classify brainwaves. Conventional machine learning models generally first use artificially designed feature extraction methods to extract features from brainwaves, which can be determined by, for example, statistical methods (e.g. averages, standard deviations, etc.), spectral components, etc. Then, the trained machine learning model, such as support vector machines, linear discriminant analysis, decision trees, random forests, or any of other algorithms, is used for classification, thereby determining the current mental activity state of the subject.

The deep learning model collects, in advance, a large amount of brainwave data of attention and rest, and uses these data to train neural networks so as to classify brainwaves. Regarding the neural network, in addition to classifying features extracted by artificially designed feature extraction methods, it can also directly perform feature learning and classifying with the time domain signals so as to determine the current mental activity state of the subject.

Furthermore, the brain-computer interface (BCI) technology, like EEG technology, has been used in many applications in people's daily life. For example, before 2000, BCI was mainly used in some controls of assistive devices, cognitive functions, learnings, rehabilitation, and the likes. After 2010, the applications of BCI become more life-oriented. For example, it can be used to detect emotions, mental states, etc., and can combine general usage and entertainment, such as wheelchair operation and video games. In 2020, multiple BCIs have been integrated for more applications.

As mentioned above, when BCI technology is applied to the control of assistive device, it is namely the interface between the brain and the machine (or computer). The overall concept thereof is to use specific detection methods to capture brain signals and to analyze and translate them into computer instructions, so that the computer can execute the instructions to operate external machines so as to achieve certain control functions. In addition, when the BCI is applied to cognitive functions (e.g. for training user's attention), it captures brain signals through specific detection methods and analyzes the user's attention level. In addition to the above-mentioned various applications, the BCI technology has also been applied to language learning, which mainly uses attention mechanism to improve learning effect.

Although EEG and BCI technologies have been widely used in various fields, most of them are limited to single-function applications. So far, there is no method or device relating to recover the object initial information into the object expectation information by analyzing the brainwave signals generated based on user's mental activity and judging the classification or classification intensity of the mental activity, wherein the process of recovering the object expectation information can achieve mental training effect and multi-functional application effect. In other words, it is desired to provide a method and device that can recover the object initial information into the object expectation information by analyzing and judging the brainwave signals generated based on user's mental activity, thereby achieving mental training effect and multi-functional application effect.

### SUMMARY

In view of the foregoing, an objective of this disclosure is to provide a method and device that can analyze the brainwave signals generated based on user's mental activity and judge the classification or classification intensity of the mental activity, and then recover the object initial information into the object expectation information based on the classification or classification intensity, thereby achieving mental training effect and multi-functional application effect.

To achieve the above, this disclosure provides a device for executing a method for performing recovery functions, used in conjunction with a brainwave detector, which includes a display unit, a brainwave signal receiving module, an analysis and judgment unit and a recover control unit. The display unit is configured to display at least one object initial information of an application program. The brainwave signal receiving module receives at least one brainwave signal from the brainwave detector. The analysis and judgment unit analyzes and judges the at least one brainwave signal to generate at least one mental activity classification information. The recover control unit generates alteration information based on the mental activity classification information, recovers the object initial information into object expectation information according to the alteration information, and displays the object expectation information on the display unit.

In one embodiment, the brainwave signal receiving module receives at least two of the brainwave signals; the analysis and judgment unit analyzes and judges the brainwave signals respectively, and generates at least two of the mental activity classification information for the at least two brainwave signals respectively; and the recover control unit generates the alteration information based on the at least two of the mental activity classification information.

In one embodiment, the analysis and judgment unit judges a mental activity classification of the brainwave signal by analyzing brainwave pattern information or brainwave characteristic information of the brainwave signal, and generates the mental activity classification information according to the mental activity classification. The mental activity classification information includes an attention information or a rest (non-attention) information, or includes an attention intensity information or a rest (non-attention) intensity information.

In one embodiment, the device further includes a memory unit, which is configured to store the brainwave signal.

In one embodiment, the device further includes a sound module, which is configured to output a sound related to the object expectation information.

In one embodiment, the object initial information or the object expectation information includes a text information, a pattern information, an image information, a symbol information, a sound information, or a combination thereof.

In one embodiment, the analysis and judgment unit analyzes and judges the brainwave signal by using a spectrum analysis model, a signal analysis model, a machine learning model or a deep learning model.

To achieve the above, this disclosure also provides a method for performing recovery functions with a device, includes the following steps of: a first step of configuring a display unit to display at least one object initial information of an application program; a second step of receiving at least one brainwave signal; a third step of analyzing and judging the at least one brainwave signal to generate at least one mental activity classification information; and a fourth step of generating alteration information based on the mental activity classification information, recovering the object initial information into object expectation information according to the alteration information, and displaying the object expectation information on the display unit.

In one embodiment, in the second step, the brainwave signal receiving module receives at least two of the brainwave signals; in the third step, the analysis and judgment unit analyzes and judges brainwave pattern information or brainwave characteristic information of the at least two brainwave signals respectively so as to determine mental activity classifications of the brainwave signals, and generates at least two of the mental activity classification information for the at least two brainwave signals respectively; and in the fourth step, the recover control unit generates the alteration information based on the at least two of the mental activity classification information.

As mentioned above, the device and method of this disclosure have an analysis and judgment unit for judging the brainwave signal generated based on the mental activity of a user thereby generating the mental activity classification information, so that the user can realize the training result after the mental activity according to the metal activity classification information. In addition, the device and method of this disclosure have a recover control unit for generating alteration information and recovering the object initial information into object expectation information according to the alteration information, so that the user may utilize the device and method of this disclosure in multi-functional applications. In other words, the device and method of this disclosure can achieve both mental training effect and multi-functional application effect.

### BRIEF DESCRIPTION OF THE DRAWINGS

The disclosure will become more fully understood from the detailed description and accompanying drawings, which are given for illustration only, and thus are not limitative of the present disclosure, and wherein:

FIG. 1 is a block diagram of the device of this disclosure;

FIG. 2A is a schematic diagram showing the display unit displayed with object initial information and mental activity classification information;

FIG. 2B is a schematic diagram showing the display unit displayed with object expectation information and mental activity classification information;

FIG. 3A is a schematic diagram showing a brainwave signal graph obtained by measuring the prefrontal lobe, wherein the user is under a non-attention state;

FIG. 3B is a schematic diagram showing a brainwave signal graph obtained by measuring the prefrontal lobe, wherein the user is under an attention state;

FIG. 4A is a schematic diagram showing the display unit displayed with another object initial information and mental activity classification information;

FIG. 4B is a schematic diagram showing the display unit displayed with another object expectation information and mental activity classification information;

FIG. 5A is a schematic diagram showing the display unit displayed with another object initial information and mental activity classification information;

FIG. 5B is a schematic diagram showing the display unit displayed with another object expectation information and mental activity classification information;

FIG. 6 is another block diagram of the device of this disclosure;

FIG. 7 is another block diagram of the device of this disclosure;

FIG. 8A is a schematic diagram showing the display unit displayed with another object initial information and mental activity classification information;

FIG. 8B is a schematic diagram showing the display unit displayed with another object expectation information and mental activity classification information;

FIG. 9A is a schematic diagram showing a brainwave signal graph obtained by measuring the prefrontal lobe, wherein the user is under a non-attention state;

FIG. 9B is a schematic diagram showing a brainwave signal graph obtained by measuring the prefrontal lobe, wherein the user is under a medium attention state;

FIG. 9C is a schematic diagram showing a brainwave signal graph obtained by measuring the prefrontal lobe, wherein the user is under a fully attention state;

FIG. 10 shows a spectrum diagram obtained by performing spectral conversions on the brainwave signals shown in FIGS. 9A to 9C;

FIG. 11 is a schematic diagram showing the display unit displayed with object initial information, which is originally the object expectation information as shown in FIG. 8B, as well as the next object expectation information and mental activity classification information;

FIG. 12 is a schematic diagram showing the display unit displayed with object initial information, which is originally the object expectation information as shown in FIG. 8B, as well as the intermediate alteration information and mental activity classification information;

FIG. 13 is a flow chart of the method of this disclosure; and

FIG. 14 is another flow chart of the method of this disclosure.

### DETAILED DESCRIPTION OF THE DISCLOSURE

Before describing the specific embodiments of the present disclosure, it should be noted that in the embodiments of the present disclosure, the terms “unit” and “module” used refer to a unit that performs at least one function or operation. The unit may be implemented as hardware or software, or a combination of hardware and software. Throughout this specification, “application” or “application program” may refer to a collection of computer programs designed to execute predetermined operations.

The applications described in this specification may vary according to one or more exemplary embodiments. For example, applications may include, for example but not limited to, webpage browser applications, dictionary applications, translation applications, music player applications, video player applications, messaging applications, map applications, image data applications, game applications, sports support applications, language learning applications, music learning applications, puzzle applications, and picture or image reconstruction applications.

In this application, the device 1 described may include, for example but not limited to, a desktop computer, a mobile phone, a smart phone, a laptop computer, a tablet personal computer (PC), an e-book terminal, a personal digital assistant (PDA), a navigation device, an Internet protocol television (IPTV), a digital television (DTV), or a consumer electronics (CE) device (e.g. a refrigerator with display unit).

In this application, the object initial information or the object expectation information in the following content includes a text information, a pattern information, an image information, a symbol information, a sound information, or a combination of two or more of the above.

In this application, the mental activity in the following content refers to the state of the user or subject's brainwaves, and the mental activity classification information refers to the classification information of the user or subject's state, including the attention state or rest state, after analyzing and judging the brainwave signal, or the attention intensity or the rest intensity obtained by further quantifying the information on the attention state or rest state.

In this application, the alteration information in the following content includes information that is added to or subtracted from the object initial information, or information that is altered from the object initial information. It should be noted that although the embodiments in this application all use the information added to the object initial information as an example, the alteration information certainly includes the information subtracted from the object initial information and the information altered from the object initial information.

In this application, the non-attention in the following content is equivalent to rest, and the medium attention refers to the attention state between rest (non-attention) and fully attention.

The present disclosure will be apparent from the following detailed description, which proceeds with reference to the accompanying drawings, wherein the same references relate to the same elements.

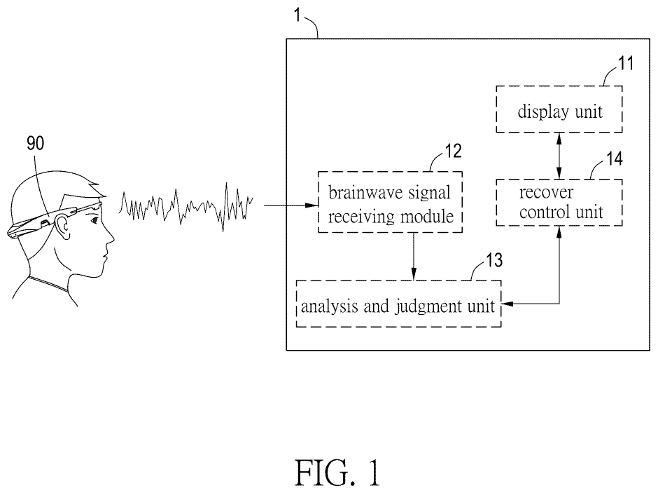

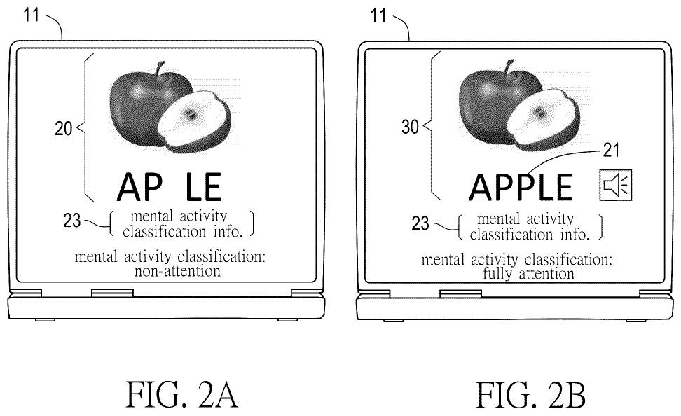

Referring to FIG. 1, a device 1 for performing recovery functions of the embodiment of this application includes a display unit 11, a brainwave signal receiving module 12, an analysis and judgment unit 13, and a recover control unit 14. Referring to FIG. 2A, when the user starts to use the device 1, the display unit 11 firstly shows an object initial information 20. As shown in FIG. 2A, the shown object initial information 20 includes a picture of apple and an incomplete word “AP LE”. Referring to FIG. 1 again, the brainwave signal receiving module 12 is configured to receive at least one brainwave signal, which is detected by the brainwave detector 90. In this embodiment, the brainwave signal receiving module 12 may receive the brainwave signal by wired connection or wireless connection.

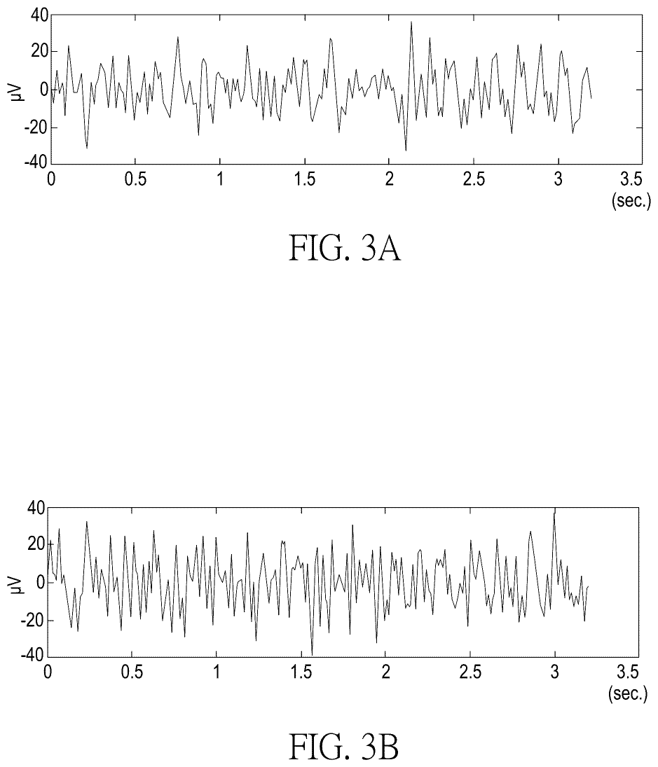

After the brainwave signal receiving module 12 receives the user's brainwave signal, the received brainwave signal will be transmitted to the analysis and judgment unit 13 for further analysis and judgment. As shown in FIGS. 3A and 3B, if the analyzing result of the analysis and judgment unit 13, after analyzing the user's brainwave signal characteristics (density or high/low frequency components), is relatively similar to the waveform as shown in FIG. 3A, it determines that the user is in a rest state. In another case, if the analyzing result of the analysis and judgment unit 13, after analyzing the user's brainwave signal characteristics, is relatively similar to the waveform as shown in FIG. 3B, it determines that the user is in an attention state. In other words, the mental activity classification information 23 generated by the analysis and judgment unit 13 indicates an attention state (see FIG. 2A) or a of rest state (see FIG. 2B). Afterwards, if the user is initially set to recover the object initial information 20 in an attention state, and the analysis and judgment unit 13 determines that the user is in an attention state, the recover control unit 14 will generate an alteration information 21 (i.e., the information of “P”) accordingly, and then recover the object initial information 20 to an object expectation information 30 according to the alteration information 21. In other words, the object initial information 20 can be recovered to the object expectation information 30 through the recover control unit 14, and the object expectation information 30 is displayed on the display unit 11. As shown in FIG. 2B, the displayed object expectation information 30 at this time includes a picture of apple and a complete word “APPLE”. To be noted, the user may be initially set in a rest state to recover the object initial information 20.

As mentioned above, the analysis and judgment unit 13 can analyze the density or high/low frequency components of the user's brainwave signal through spectrum analysis so as to determine the classification of the brainwave signal. In another case, the analysis and judgment unit 13 can also analyze two frequency bands of alpha and beta waves in the brainwave signal, and then judge the user's mental activity classification intensity based on the ratio of the two frequency bands. In more detailed, the user's mental activity classification intensity can be determined based on the ratio T obtained by dividing the beta wave intensity by the alpha wave intensity in the brainwave signal. In other words, the above-mentioned mental activity classification information 23 generated by the analysis and judgment unit 13 can also be considered as the attention intensity information or rest intensity information.

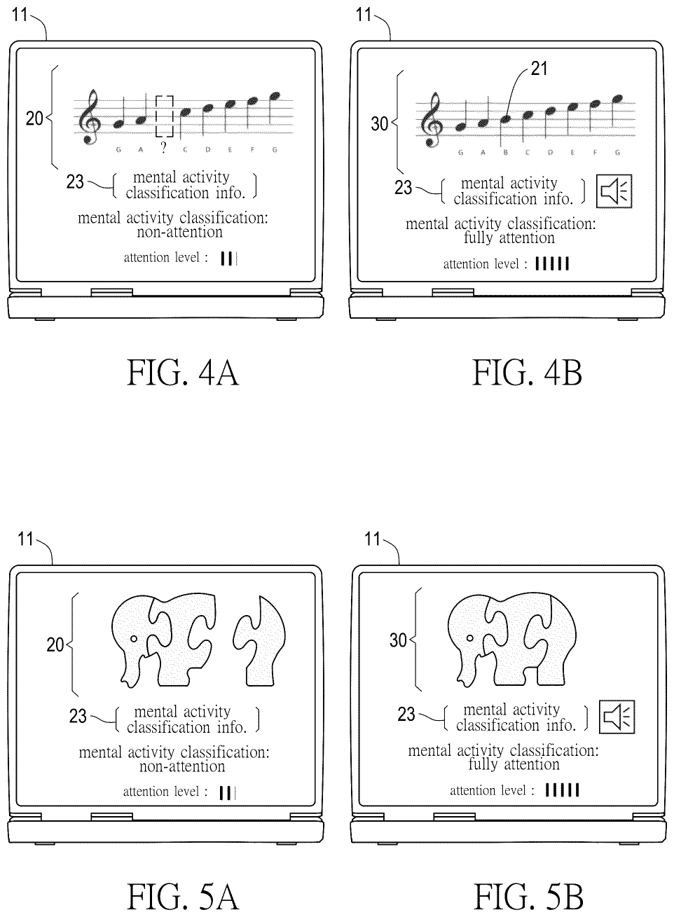

As shown in FIG. 4A, when the user starts to use the device 1 of the present disclosure, the display unit 11 firstly displays an object initial information 20. In this case, the object initial information 20 includes a five-line staff and an incomplete musical notes. In this embodiment, if the user is initially set to recover the object initial information 20 in an attention state, and the analysis and judgment unit 13 determines that the user is in an attention state and the attention intensity reaches or exceeds a threshold value Tg, then the object initial information 20 can be recovered to the object expectation information 30 based on the alteration information 21 (i.e., a note information) generated by the recover control unit 14. Then, the object expectation information 30 can be displayed on the display unit 11. As shown in FIG. 4B, the object expectation information 30 includes a five-line staff and a complete musical notes.

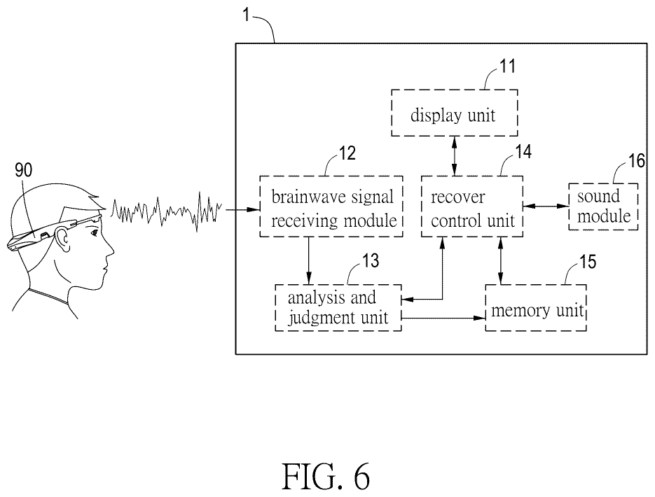

As shown in FIG. 5A, when the user starts to use the device 1 of the present disclosure, the display unit 11 firstly displays an object initial information 20. In this case, the object initial information 20 includes a plurality of puzzle pieces. In this embodiment, if the user is initially set to recover the object initial information 20 in an attention state, and the analysis and judgment unit 13 determines that the user is in an attention state and the attention intensity reaches or exceeds a threshold value Tg, then the object initial information 20 can be recovered to the object expectation information 30 based on the alteration information 21 (i.e., the information of the last puzzle piece) generated by the recover control unit 14. Then, the object expectation information 30 can be displayed on the display unit 11. As shown in FIG. 5B, the object expectation information 30 includes a completed puzzle of an elephant.

With reference to FIGS. 2A, 2B, 4A, 4B, 5A and 5B, in addition to training the user's attention, the device 1 for performing recovery functions according to the embodiments of the present disclosure can also train the user's language ability, music ability, puzzle ability, etc. at the same time. To be noted, the device 1 of the embodiment can also achieve the learning effects of any other abilities by training the suitable mental activities.

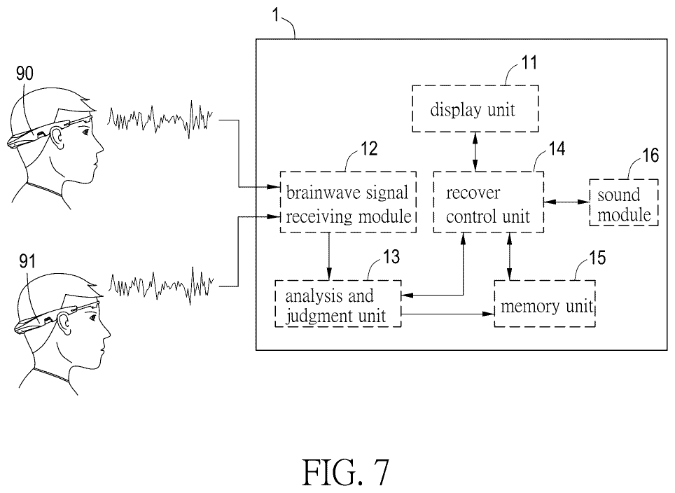

Referring to FIG. 6, the device 1 for performing recovery functions of the embodiment of this application can further include a memory unit 15 and a sound module 16. The memory unit 15 is configured to store the brainwave signal or the mental activity classification information 23. The sound module 16 is configured to output a sound related to the object expectation information 30. For example, the sound module 16 may output a sound of “APPLE” in the embodiment of FIG. 2B, or a complete musical notes in the embodiment of FIG. 4B.

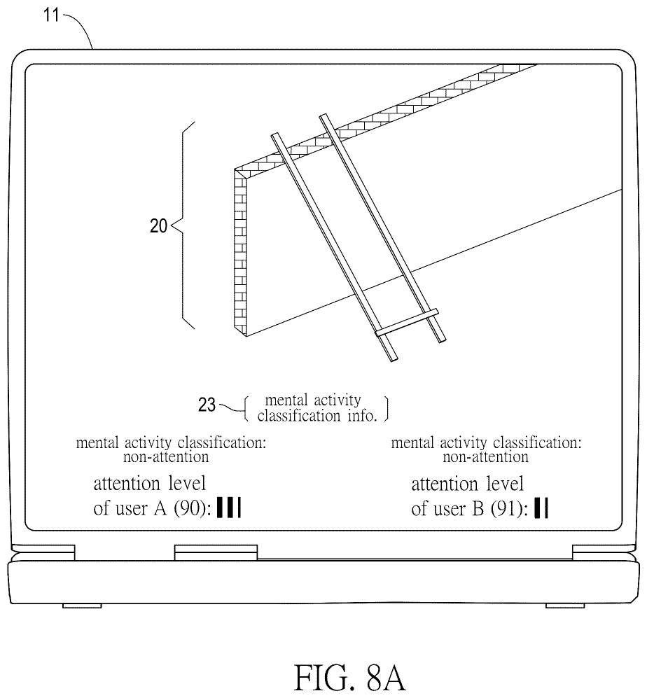

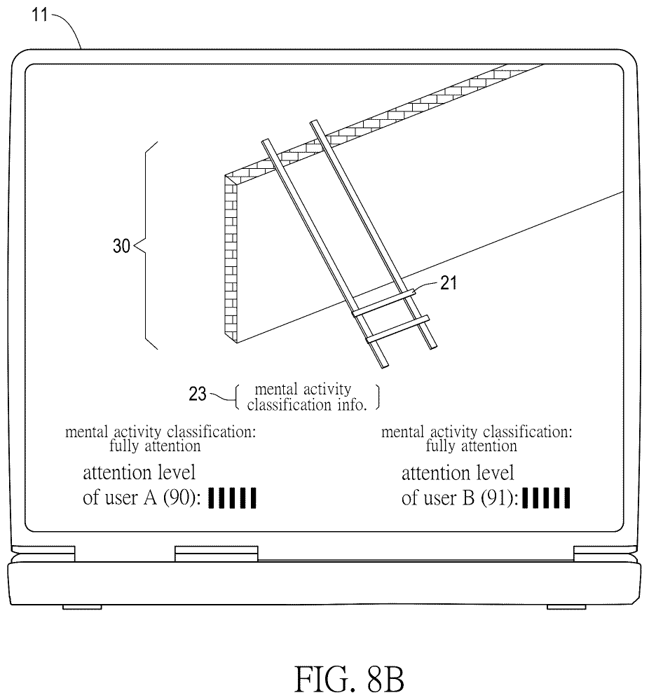

With reference to FIG. 7, in another embodiment of the present disclosure, the brainwave signal receiving module 12 of the device 1 for performing recovery functions can simultaneously receive the brainwaves measured by two brainwave detectors 90 and 91. The two brainwave signals are simultaneously sent to the analysis and judgment unit 13 for analyzing and judging. As shown in FIGS. 8A and 8B, if the analysis and judgment unit 13 respectively analyzes the brainwave signal characteristics (density or high/low frequency components) of the two brainwave signals and determines that they are relatively similar to the waveform as shown in FIG. 3A, then it judges that both users are in a rest state. In another case, if the analysis and judgment unit 13 respectively analyzes the brainwave signal characteristics of the two brainwave signals and determines that they are relatively similar to the waveform as shown in FIG. 3B, then it judges that both users are in an attention state. In other words, the mental activity classification information 23 generated by the analysis and judgment unit 13 is an attention state or a rest state. In this case, if the two users are initially set to use the attention state to recover the object initial information 20, and the analysis and judgment unit 13 determines that they are both in the attention state, an alteration information 21 is generated by the recover control unit 14, and the object initial information 20 is recovered to the object expectation information 30 based on the alteration information 21 (i.e., the second-step information). That is, as shown in FIG. 8B, the recover control unit 14 can recover the object initial information 20 to the object expectation information 30. Then, the object expectation information 30 is displayed on the display unit 11. In more detailed, the object initial information 20 as shown in FIG. 8A including a wall and a ladder with one step is recovered to the object expectation information 30 as shown in FIG. 8B including a wall and a ladder with two steps. To be noted, the user may be initially set in a rest state to recover the object initial information 20.

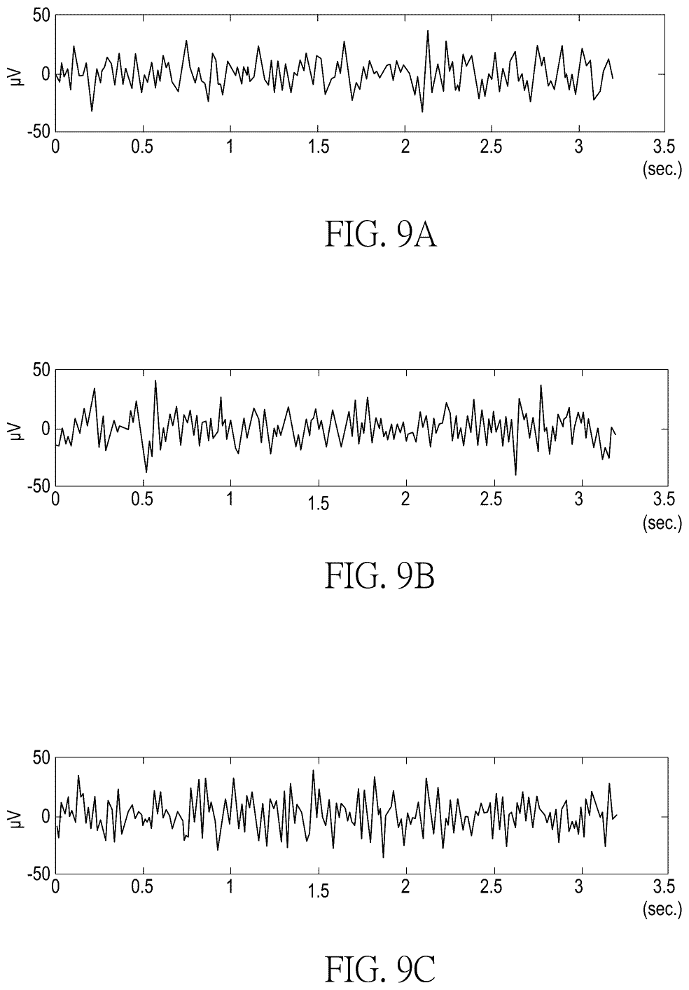

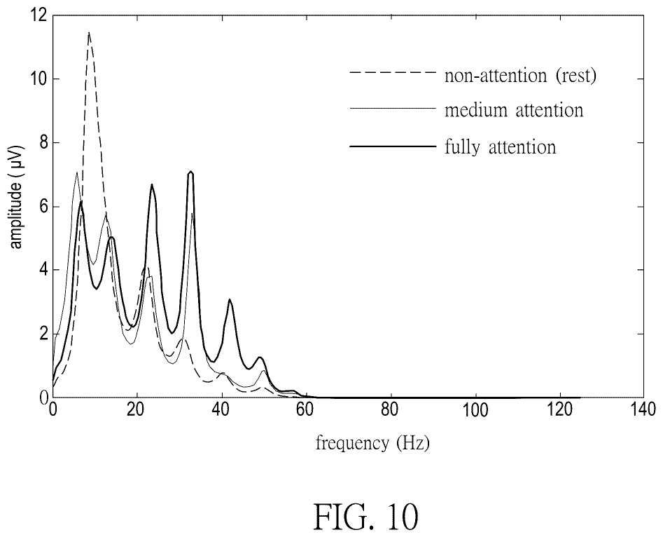

In the above embodiment, the mental activity classification information 23 generated by the analysis and judgment unit 13 can be attention intensity information or rest intensity information. The method of calculating the attention intensity or rest intensity will be further described hereinafter. If a section of the brainwave signal (e.g. a section of 3.5 seconds in the brainwave signal) is extracted, and then a filter is applied to the extracted section to remove high-frequency and low-frequency noises with retaining the section of brainwave signal of 2.5 Hz˜50 Hz, then the brainwave signals can be obtained as shown in FIGS. 9A to 9C based on the user's mental activity state. FIG. 9A shows the brainwave signal of the user in a non-attention state (i.e., rest state), FIG. 9B shows the brainwave signal of the user in a medium attention state, and FIG. 9C shows the brainwave signal of the user in a fully attention state. In this case, the brainwave signals as shown in FIGS. 9A to 9C can be processed by spectrum conversion to obtain the spectrum diagram as shown in FIG. 10. In FIG. 10, the dotted line indicates the spectrum of the rest state, the thin solid line indicates the spectrum of the medium attention state, and the thick solid line indicates the spectrum of the fully attention state. Different frequency components in the brainwave signal (e.g. the frequency components of the two frequency bands of alpha wave and beta wave) can be analyzed from the spectrum diagram as shown in FIG. 10. In other words, based on the spectrum diagram as shown in FIG. 10, the areas under the curves within the ranges of the alpha wave (8˜13 Hz) and the beta wave (13˜30 Hz) are calculated so as to obtain the quantities of the two frequency components of the alpha wave and beta wave respectively. Then, the aforementioned ratio T (attention index) can be obtained by dividing the beta wave intensity by the alpha wave intensity. For example, after calculation, in FIG. 10, the brainwave signal indicated by dotted line has T=0.7127, the brainwave signal indicated by thin solid line has T=1.0940, and the brainwave signal indicated by thick solid line has T=2.0081. If the attention threshold Tg is set to 2.0, the T values of the measured brainwave signals can be compared with the attention threshold Tg so as to obtain the mental activity classification information 23, and the intensities of the mental activity classification information 23 can be displayed on the display unit 11.

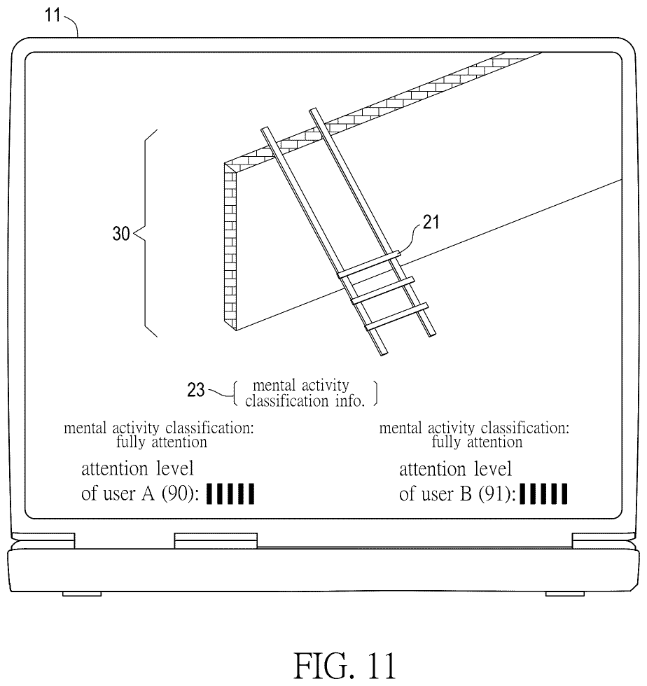

To be noted, in this embodiment, the object expectation information 30 can be used as the object initial information 20 for the next mental activity. Referring to FIGS. 8A, 8B and 11 again, when the functions shown in FIGS. 8A, 8B and 11 belong to the same application, the wall and the ladder with one step as shown in FIG. 8A indicate the object initial information 20 of the current mental activity, and the wall and the ladder with two steps as shown in FIG. 8B indicate the object expectation information 30 of the current mental activity with respect to the object initial information 20 as shown in FIG. 8A. Afterwards, if the user continues to perform the next mental activity, the object expectation information 30 of the mental activity as shown in FIG. 8B can be used as the object initial information 20 for the next mental activity as shown in FIG. 11.

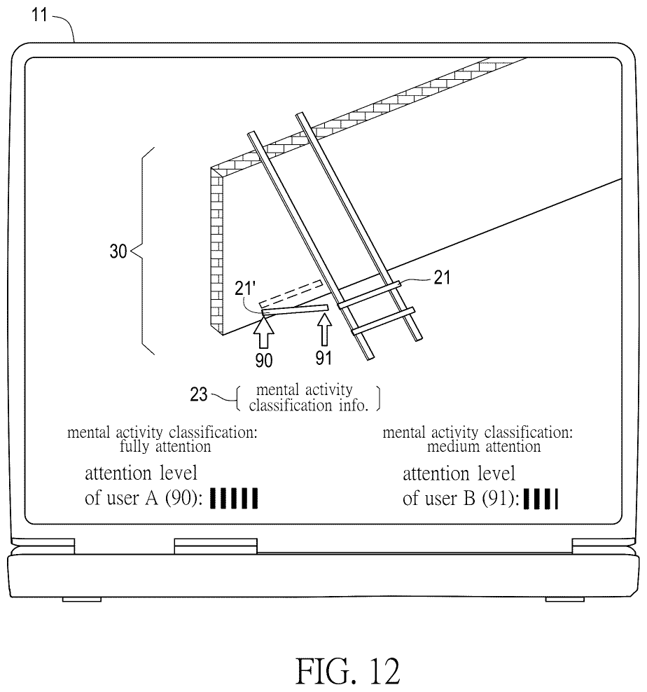

Referring to FIGS. 7, 8B and 12, when two brainwave signals are simultaneously transmitted to the analysis and judgment unit 13 for analysis and judgment, if the brainwave information of one user (i.e., from the brainwave detector 90) reaches the attention threshold Tg, and the brainwave information of the other user (i.e., from the brainwave detector 91) does not reach the attention threshold Tg, the recover control unit 14 will generate an intermediate alteration information 21′, and the intermediate alteration information 21′ can be displayed on the display unit 11.

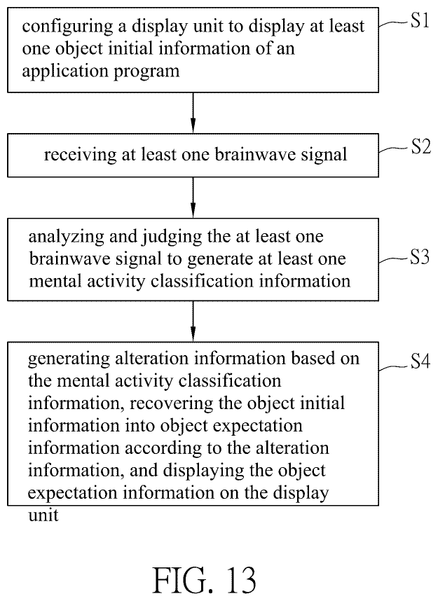

The above is a detailed description of the device 1 for performing recovery functions according to the embodiments of the present disclosure. The method for performing recovery functions with the device 1 according to an embodiment of the present disclosure will be described hereinafter with reference to FIG. 13.

As shown in FIG. 13, the method for performing recovery functions with a device includes the following steps of: a first step S1 of configuring a display unit to display at least one object initial information of an application program; a second step S2 of receiving at least one brainwave signal; a third step S3 of analyzing and judging the at least one brainwave signal to generate at least one mental activity classification information; and a fourth step S4 of generating alteration information based on the mental activity classification information, recovering the object initial information into object expectation information according to the alteration information, and displaying the object expectation information on the display unit.

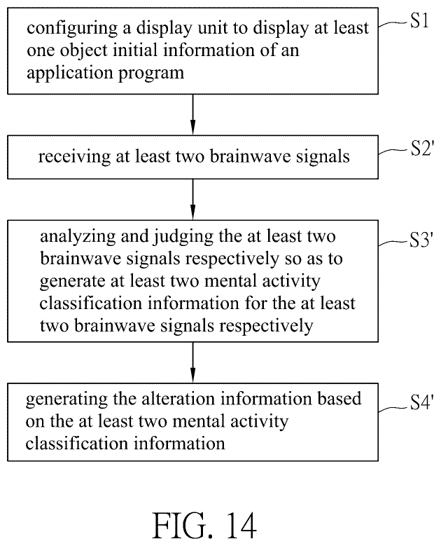

In another embodiment as shown in FIG. 14, the second step S2, the third step S3, and the fourth step S4 can be substituted by a second step S2′, a third step S3′, and a fourth step S4′. In this embodiment, the second step S2′ is to receive at least two brainwave signals, the third step S3′ is to analyze and judge the at least two brainwave signals respectively so as to generate at least two mental activity classification information for the at least two brainwave signals respectively, and the fourth step S4′ is to generate the alteration information based on the at least two mental activity classification information.

To be noted, other related technical descriptions of the method for performing a recovery functions with a device according to the embodiment of the present disclosure are the same as or similar to the descriptions of the above-mentioned device 1, so the detailed descriptions thereof will be omitted.

In summary, the device and method of this disclosure have an analysis and judgment unit for judging the brainwave signal generated based on the mental activity of a user thereby generating the mental activity classification information, so that the user can realize the training result after the mental activity according to the metal activity classification information. In addition, the device and method of this disclosure have a recover control unit for generating alteration information and recovering the object initial information into object expectation information according to the alteration information, so that the user may utilize the device and method of this disclosure in multi-functional applications. In other words, the device and method of this disclosure can achieve both mental training effect and multi-functional application effect.

Although the disclosure has been described with reference to specific embodiments, this description is not meant to be construed in a limiting sense. Various modifications of the disclosed embodiments, as well as alternative embodiments, will be apparent to persons skilled in the art. It is, therefore, contemplated that the appended claims will cover all modifications that fall within the true scope of the disclosure.

What is claimed is:

1. A device for executing a method for performing recovery functions, used in conjunction with a brainwave detector, comprising:
a display unit configured to display at least one object initial information of an application program;
a brainwave signal receiving module for receiving at least one brainwave signal from the brainwave detector;
a memory unit configured to store the brainwave signal;
an analysis and judgment unit analyzing and judging brainwave pattern information or brainwave characteristic information of the at least one brainwave signal so as to determine a mental activity classification of the brainwave signal, and generating at least one mental activity classification information according to the mental activity classification, wherein the mental activity classification information comprises an attention information or a rest information, or the mental activity classification information comprises an attention intensity information or a rest intensity information; and
a recover control unit generating alteration information based on the mental activity classification information, recovering the object initial information into object expectation information according to the alteration information, and displaying the object expectation information on the display unit;
wherein, the alteration information comprises increased or decreased information for the object initial information; and
wherein, the analysis and judgment unit analyzes and judges the brainwave signal by using a spectrum analysis model, a signal analysis model, a machine learning model or a deep learning model.

2. The device of claim 1, wherein the brainwave signal receiving module receives at least two of the brainwave signals; the analysis and judgment unit analyzes and judges the brainwave signals respectively, and generates at least two of the mental activity classification information for the at least two brainwave signals respectively; and the recover control unit generates the alteration information based on the at least two of the mental activity classification information.

3. The device of claim 1, wherein the device further comprises a sound module, which is configured to output a sound related to the object expectation information.

4. The device of claim 1, wherein the object initial information or the object expectation information comprises a text information, a pattern information, an image information, a symbol information, a sound information, or a combination thereof.

5. A method for performing recovery functions with a device, comprising:
a first step of configuring a display unit to display at least one object initial information of an application program;
a second step of receiving at least one brainwave signal by a brainwave signal receiving module, wherein the brainwave signal is stored in a memory unit;
a third step of analyzing and judging brainwave pattern information or brainwave characteristic information of the at least one brainwave signal by an analysis and judgment unit so as to determine a mental activity classification of the brainwave signal, and generating at least one mental activity classification information according to the mental activity classification, wherein the mental activity classification information comprises an attention information or a rest information, or the mental activity classification information comprises an attention intensity information or a rest intensity information; and
a fourth step of generating alteration information based on the mental activity classification information by a recover control unit, recovering the object initial information into object expectation information according to the alteration information, and displaying the object expectation information on the display unit;
wherein, the alteration information comprises increased or decreased information for the object initial information; and
wherein, the analysis and judgment unit analyzes and judges the brainwave signal by using a spectrum analysis model, a signal analysis model, a machine learning model or a deep learning model.

6. The method of claim 5, wherein in the second step, the brainwave signal receiving module receives at least two of the brainwave signals; in the third step, the analysis and judgment unit analyzes and judges brainwave pattern information or brainwave characteristic information of the at least two brainwave signals respectively so as to determine mental activity classifications of the brainwave signals, and generates at least two of the mental activity classification information for the at least two brainwave signals respectively; and in the fourth step, the recover control unit generates the alteration information based on the at least two of the mental activity classification information.

7. The method of claim 5, wherein a sound related to the object expectation information is outputted by a sound module.

8. The method of claim 5, wherein the object initial information or the object expectation information comprises a text information, a pattern information, an image information, a symbol information, a sound information, or a combination thereof.
